(19) United States
(12) Patent Application Publication
(10) Pub. No.: US 2007/0232590 A1
(43) Pub. Date: Oct. 4, 2007

(54) 3-Cylcoalkylbenzazepines as Histamine H3 Antagonists

(75) Inventors: Mark James Bamford, Harlow (GB); Paula Louise Pickering, Harlow (GB); David Matthew Wilson, Harlow (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, GREENFORD, MIDDLESEX (GB)

(21) Appl. No.: 11/570,383

(22) PCT Filed: Jun. 16, 2005
(86) PCT No.: PCT/EP05/06861

§ 371 Date: Dec. 11, 2006

(30) Foreign Application Priority Data
Jun. 18, 2004 (GB) 0413770.9
Jun. 18, 2004 (GB) 0413764.2
Jun. 18, 2004 (GB) 0413766.7
Jun. 18, 2004 (GB) 0413765.9
Jun. 18, 2004 (GB) 0413757.6
Jun. 18, 2004 (GB) 0413758.4
Jun. 18, 2004 (GB) 0413769.1
Jun. 18, 2004 (GB) 0413768.3
Jun. 18, 2004 (GB) 0413763.4

(51) Int. Cl. A61K 31/55; C07D 233/16

(52) U.S. Cl. 514/217.01; 540/594

(57) ABSTRACT

The present invention relates to novel benzazepine derivatives having pharmacological activity, processes for their preparation, to compositions containing them and to their use in the treatment of neurological and psychiatric disorders.

The present invention relates to novel benzazepine derivatives having pharmacological activity, processes for their preparation, to compositions containing them and to their use in the treatment of neurological and psychiatric disorders.

JP 2001226269 and WO 00/23437 (Takeda Chem Ind Ltd) describe a series of benzazepine derivatives which are claimed to be useful in the treatment of obesity. DE 2207430, U.S. Pat. No. 4,210,749 and FR 2171879 (Pennwalt Corp) and GB 1268243 (Wallace and Tiernan Inc) all describe a series of benzazepine derivatives which are claimed as being antagonists for narcotics (such as morphine or codeine) and also anti-histamines and anticholinergic agents. WO 02/14513 (Takeda Chem Ind Ltd) describe a series of benzazepine derivatives with GPR12 activity which are claimed to be useful in the treatment of attention deficit disorder, narcolepsy or anxiety. WO 02/02530 (Takeda Chem Ind Ltd) describe a series of benzazepine derivatives as GPR14 antagonists which are claimed to be useful in the treatment of hypertension, atherosclerosis and cardiac infarction. WO 01/03680 (Isis Innovation Ltd) describe a series of benzazepine derivatives which are claimed as effective agents in the preparation of cells for transplantation in addition to the inhibition of diseases such as diabetes. WO 00/21951 (SmithKline Beecham plc) discloses a series of tetrahydrobenzazepine derivatives as modulators of dopamine D3 receptors which are claimed to be useful as antipsychotic agents. WO 01/87834 (Takeda Chem Ind Ltd) describe a series of benzazepine derivatives as MCH antagonists which are claimed to be useful in the treatment of obesity. WO 02/15934 (Takeda Chem Ind Ltd) describe a series of benzazepine derivatives as urotensin II receptor antagonists which are claimed to be useful in the treatment of neurodegenerative disorders. WO 04/018432 (Eli Lilly and Company) describe a series of substituted azepines as histamine H3 receptor antagonists. WO 2004/05639 (Glaxo Group Ltd.; published 8 Jul. 2004) describes a series of benzazepine derivatives and their use in the treatment of neurological disorders. WO 03/090751 (Pfizer Products Inc.) discloses a series of N-substituted heteroaryloxy-aryloxy-2,4,6-trione metalloproteinase inhibitors. The compounds are claimed to be useful in the treatment of inflammation, cancer and other disorders. JP 63094239 (Konishiroku Photo KK) discloses the use of benzazepine derivatives in photographic materials. WO 2004/026303 (Eli Lilly and Company) describes a series of diaryl ethers as opioid receptor antagonists. The compounds are disclosed to be useful in the treatment of obesity.

The histamine H3 receptor is predominantly expressed in the mammalian central nervous system (CNS), with minimal expression in peripheral tissues except on some sympathetic nerves (Leurs et al., (1998), Trends Pharmacol. Sci. 19, 177-183). Activation of H3 receptors by selective agonists or histamine results in the inhibition of neurotransmitter release from a variety of different nerve populations, including histaminergic and cholinergic neurons (Schlicker et al., (1994), Fundam. Clin. Pharmacol. 8, 128-137). Additionally, in vitro and in vivo studies have shown that H3 antagonists can facilitate neurotransmitter release in brain areas such as the cerebral cortex and hippocampus, relevant to cognition (Onodera et al., (1998), In: The Histamine H3 receptor, ed Leurs and Timmerman, pp 255-267, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al., (1999), Behav. Brain Res. 104, 147-155). These data suggest that novel H3 antagonists and/or inverse agonists such as the compounds of the present invention could be useful for the treatment of cognitive impairments in neurological diseases such as Alzheimer's disease and related neurodegenerative disorders.

The present invention provides, in a first aspect, a compound which is 1-{6-[(3-cyclopentyl-2,3,4,5-tetrahydro-1H-3-benzazepin-7-yl)oxy]-3-pyridinyl}-2-pyrrolidinone or a pharmaceutically acceptable salt thereof.

In a second aspect, the present invention provides a compound which is 1-{5-[(3-cyclopentyl-2,3,4,5-tetrahydro-1H-3-benzazepin-7-yl)oxy]-2-pyrazinyl}-2-pyrrolidinone or a pharmaceutically acceptable salt thereof.

In a third aspect, the present invention provides a compound which is 1-{4-[(3-cyclobutyl-2,3,4,5-tetrahydro-1H-3-benzazepin-7-yl)oxy]-3-fluorophenyl}-3-methyl-2-imidazolidinone or a pharmaceutically acceptable salt thereof.

In a fourth aspect, the present invention provides a compound which is 1-{4-[(3-cyclobutyl-2,3,4,5-tetrahydro-1H-3-benzazepin-7-yl)oxy]phenyl}-2-pyrrolidinone or a pharmaceutically acceptable salt thereof.

In a fifth aspect, the present invention provides a compound which is 3-{6-[(3-cyclopentyl-2,3,4,5-tetrahydro-1H-3-benzazepin-7-yl)oxy]-3-pyridazinyl}-1,3-oxazolidin-2-one or a pharmaceutically acceptable salt thereof.

In a sixth aspect, the present invention provides a compound which is 1-{6-[(3-cyclopentyl-2,3,4,5-tetrahydro-1H-3-benzazepin-7-yl)oxy]-3-pyridazinyl}-2-pyrrolidinone or a pharmaceutically acceptable salt thereof.

In a seventh aspect, the present invention provides a compound which is 3-cyclopentyl-7-{[5-(3-methyl-1,2,4-oxadiazol-5-yl)-2-pyrazinyl]oxy}-2,3,4,5-tetrahydro-1H-3-benzazepine or a pharmaceutically acceptable salt thereof.

In an eighth aspect, the present invention provides a compound which is 3-cyclobutyl-7-{[5-(3-methyl-1,2,4-oxadiazol-5-yl)-2-pyridinyl]oxy}-2,3,4,5-tetrahydro-1H-3-benzazepine or a pharmaceutically acceptable salt thereof.

In a ninth aspect, the present invention provides a compound which is 3-cyclopentyl-7-{[5-(3-methyl-1,2,4-oxadiazol-5-yl)-2-pyridinyl]oxy}-2,3,4,5-tetrahydro-1H-3-benzazepine or a pharmaceutically acceptable salt thereof.

The compounds of the present invention may form acid addition salts with acids, such as conventional pharmaceutically acceptable acids, for example maleic, hydrochloric, hydrobromic, phosphoric, acetic, fumaric, salicylic, sulphate, citric, lactic, mandelic, tartaric and methanesulphonic. Salts, solvates and hydrates of the compound of the present invention form a further aspect of the invention.

A pharmaceutically acceptable acid addition salt can be formed by reaction of the free base with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration.

The invention includes within its scope all possible stoichiometric and non-stoichiometric forms of the salts of the compounds of the invention including hydrates and solvates.

The compounds of the present invention are capable of existing in stereoisomeric forms. It will be understood that the invention encompasses all geometric and optical isomers of the compounds of the invention and mixtures thereof including racemates. Tautomers also form an aspect of the invention.

The compounds of the present invention may be described by general formula (I) as follows:

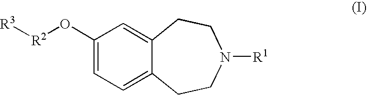

When the compound of the present invention is 1-{6-[(3-cyclopentyl-2,3,4,5-tetrahydro-1H-3-benzazepin-7-yl)oxy]-3-pyridinyl}-2-pyrrolidinone, R¹ represents cyclopentyl, R² represents pyridin-3-yl and R³ represents pyrrolidin-2-onyl.

When the compound of the present invention is 1-{5-[(3-cyclopentyl-2,3,4,5-tetrahydro-1H-3-benzazepin-7-yl)oxy]-2-pyrazinyl}-2-pyrrolidinone, R¹ represents cyclopentyl, R² represents pyrazin-3-yl and R³ represents pyrrolidin-2-onyl.

When the compound of the present invention is 1-{4-[(3-cyclobutyl-2,3,4,5-tetrahydro-1H-3-benzazepin-7-yl)oxy]-3-fluorophenyl}-3-methyl-2-imidazolidinone, R¹ represents cyclobutyl, R² represents 3-fluorophenyl and R³ represents 3-methyl-imidazolidin-2-onyl.

When the compound of the present invention is 1-{4-[(3-cyclobutyl-2,3,4,5-tetrahydro-1H-3-benzazepin-7-yl)oxy]phenyl}-2-pyrrolidinone, R¹ represents cyclobutyl, R²represents phenyl and R³ represents pyrrolidin-2-onyl.

When the compound of the present invention is 3-{6-[(3-cyclopentyl-2,3,4,5-tetrahydro-1H-3-benzazepin-7-yl)oxy]-3-pyridazinyl}-1,3-oxazolidin-2-one, R¹ represents cyclopentyl, R² represents pyridazin-3-yl and R³ represents 1,3-oxazolidin-2-onyl.

When the compound of the present invention is 1-{6-[(3-cyclopentyl-2,3,4,5-tetrahydro-1H-3-benzazepin-7-yl)oxy]-3-pyridazinyl}-2-pyrrolidinone, R¹ represents cyclopentyl, R² represents pyridazin-3-yl and R³ represents pyrrolidin-2-onyl.

When the compound of the present invention is 3-cyclopentyl-7-{[5-(3-methyl-1,2,4-oxadiazol-5-yl)-2-pyrazinyl]oxy}-2,3,4,5-tetrahydro-1H-3-benzazepine, R¹ represents cyclopentyl, R² represents pyrazin-2-yl and R³ represents 3-methyl-1,2,4-oxadiazol-5-yl.

When the compound of the present invention is 3-cyclobutyl-7-{[5-(3-methyl-1,2,4-oxadiazol-5-yl)-2-pyridinyl]oxy}-2,3,4,5-tetrahydro-1H-3-benzazepine, R¹ represents cyclobutyl, R²represents pyridin-2-yl and R³ represents 3-methyl-1,2,4-oxadiazol-5-yl.

When the compound of the present invention is 3-cyclopentyl-7-{[5-(3-methyl-1,2,4-oxadiazol-5-yl)-2-pyridinyl]oxy}-2,3,4,5-tetrahydro-1H-3-benzazepine, R¹ represents cyclopentyl, R² represents pyridin-2-yl and R³ represents 3-methyl-1,2,4-oxadiazol-5-yl.

The present invention also provides a process for the preparation of the compounds of formula (I). The process comprises:

(a) reacting a compound of formula (II)

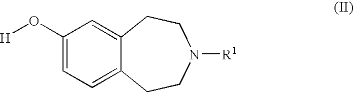

wherein R¹ is as defined above, with a compound of formula R³—R²-L¹, wherein R² and R³ are as defined above, and L¹ represents a suitable leaving group such as a halogen atom (eg. bromine or iodine); or

(b) reacting a compound of formula (III)

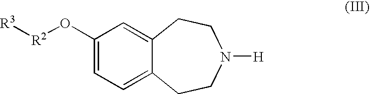

wherein R², R³ and n are as defined above, with a compound of formula R¹-L², wherein R¹ is as defined above for R¹ and L² represents a suitable leaving group such as a halogen atom (eg. bromine, iodine or tosylate); or

(c) reacting a compound of formula (III) as defined above, with a ketone of formula R^{1′}═O, wherein R¹ is cyclobutyl or cyclopentyl; or

(d) where the R³ group of the compound of formula (I) is pyrrolidin-2-onyl, 3-methyl-imidazolidin-2-onyl or 1,3-oxazolidin-2-onyl, reacting a compound of formula (IV)

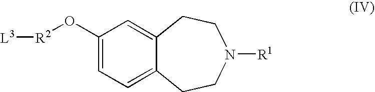

wherein R¹ and R² are as defined above and L³ represents a suitable leaving group such as a halogen atom (eg. iodine), with 2-pyrrolidinone, 1-methyl-2-imidazolidinone or 1,3-oxazolidin-2-one;

(e) where the R³ group of the compound of formula (I) is 3-methyl-1,2,4-oxadiazol-5-yl, reacting a compound of formula (V)

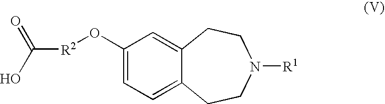

wherein R¹ and R² are as defined above with 1,1′-(Oxomethanediyl)bis-1H-imidazole; or

(f) deprotecting a compound of formula (I) which is protected.

Process (a) may be carried out as described for process (a) of WO 2004/056369.

Process (b) may be carried out as described for process (b) of WO 2004/056369.

Process (c) may be carried out as described for process (c) of WO 2004/056369.

Process (d) typically comprises the use of copper (I) iodide, potassium carbonate and N,N′-dimethyl-1,2-ethanediamine in a suitable solvent such as dry 1,4-dioxane or dry 1,2-dioxane at an elevated temperature. The reaction typically takes place in a microwave reactor.

Process (e) comprises the use of (1E)-N-hydroxyethanimidamide. The reaction takes place in a suitable solvent such as tetrahydrofuran at an elevated temperature, most typically under reflux conditions.

In process (f), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxan or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Compounds of formula (II), (III), (IV) and (V) may be prepared by the general procedures described in WO 2004/056369.

The compounds of the present invention have affinity for and are antagonists and/or inverse agonists of the histamine H3 receptor. Hence, they are believed to be of potential use in the treatment of neurological diseases including Alzheimer's disease, dementia, age-related memory dysfunction, mild cognitive impairment, cognitive deficit, epilepsy, pain of neuropathic origin including neuralgias, neuritis and back pain, and inflammatory pain including osteoarthritis, rheumatoid arthritis, acute inflammatory pain and back pain, migraine, Parkinson's disease, multiple sclerosis, stroke and sleep disorders including narcolepsy; psychiatric disorders including schizophrenia (particularly cognitive deficit of schizophrenia), attention deficit hypereactivity disorder, depression (particularly bipolar disorder) and addiction; and other diseases including obesity, asthma, allergic rhinitis, nasal congestion, chronic obstructive pulmonary disease and gastro-intestinal disorders.

Advantageously, compounds of the present invention have good stability.

Thus the invention also provides a compound of the present invention or a pharmaceutically acceptable salt thereof, for use as a therapeutic substance in the treatment or prophylaxis of the above disorders, in particular cognitive impairments in diseases such as Alzheimer's disease and related neurodegenerative disorders.

The invention further provides a method of treatment or prophylaxis of the above disorders, in mammals including humans, which comprises administering to the sufferer a therapeutically effective amount of a compound of the present invention or a pharmaceutically acceptable salt thereof.

In another aspect, the invention provides the use of a compound of the present invention or a pharmaceutically acceptable salt thereof in the manufacture of a medicament for use in the treatment of the above disorders.

When used in therapy, the compounds of the present invention are usually formulated in a standard pharmaceutical composition. Such a composition can be prepared using standard procedures.

Thus, the present invention further provides a pharmaceutical composition for use in the treatment of the above disorders which comprises a compound of the present invention or a pharmaceutically acceptable salt thereof and a pharmaceutically acceptable carrier.

The present invention further provides a pharmaceutical composition which comprises the compound of the present invention or a pharmaceutically acceptable salt thereof and a pharmaceutically acceptable carrier.

The compounds of the present invention may be used in combination with other therapeutic agents, for example histamine H1 antagonists or medicaments claimed to be useful as either disease modifying or symptomatic treatments of Alzheimer's disease. Suitable examples of such other therapeutic agents may be agents known to modify cholinergic transmission such as 5-HT₆ antagonists, M1 muscarinic agonists, M2 muscarinic antagonists or acetylcholinesterase inhibitors. When the compounds of the present invention are used in combination with other therapeutic agents, the compound and agent may be administered either sequentially or simultaneously by any convenient route.

The invention thus provides, in a further aspect, a combination comprising a compound of the present invention or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of the present invention or a pharmaceutically acceptable derivative thereof is used in combination with a second therapeutic agent active against the same disease state the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.1 to 200 mg and even more suitably 1.0 to 200 mg. In one aspect, a suitable unit dose would be 0.1-50 mg. Such unit doses may be administered more than once a day, for example two or three a day. Such therapy may extend for a number of weeks or months.

The following Descriptions and Examples illustrate the preparation of the compounds of the invention.

Description 1

### 1,1-Dimethylethyl 7-[(5-iodo-2-pyridinyl)oxy]-1,2,4,5-tetrahydro-3H-3-benzazepine-3-carboxylate (D1)

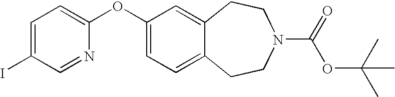

Sodium hydride (1.67 g of 60% dispersion; 41.8 mmol) was added to a solution of 1,1-dimethylethyl 7-hydroxy-1,2,4,5-tetrahydro-3H-3-benzazepine-3-carboxylate (9.2 g; 34.8 mmol, obtainable by the process described in Description 3 from WO 02/40471) in dry dimethylformamide (100 ml) and the reaction was cooled in ice/water under argon. When the addition was complete. The mixture was allowed to warm to room temperature and was stirred at room temperature for 2 hours. 2-Chloro-5-iodopyridine (10 g; 41.8 mmol) was added and the mixture heated at 95° C. for 18 hours under argon. The mixture was allowed to cool and was poured into ice/water (˜400 ml). This mixture was stirred until the ice had melted. This suspension was extracted with ethyl acetate (4×250 ml). The extracts were combined, washed (2×100 ml water, 150 ml brine) and evaporated to give a beige solid. The residue was purified on a 400 g biotage cartridge eluting with 5% and then 10% ethylacetate in pentane. Fractions containing the product were combined and evaporated to afford a white powder (13.4 g; 83%) MS (AP+), m/e 467 [M+H].

Description 1a

### 1,1-Dimethylethyl 7-[(5-bromo-2-pyridinyl)oxy]-1,2,4,5-tetrahydro-3H-3-benzazepine-3-carboxylate (D1a)

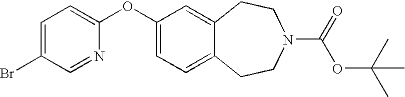

Sodium hydride (8.9 g of a 60% dispersion in mineral oil; 0.22 mol) was added portion wise to stirring solution of 1,1-dimethylethyl 7-hydroxy-1,2,4,5-tetrahydro-3H-3-benzazepine-3-carboxylate (obtainable by the process described in Description 3 from WO 02/40471) (50 g, 0.21 mol) in dry N-methylpyrrolidinone (350 ml) and the mixture stirred at room temperature for 3 hours. 5-Bromo-2-chloropyridine (44.8 g, 0.23 mol) was added portion wise and the mixture heated to an internal temperature of 97° C. and stirred under argon for 18 hours. The mixture was allowed to cool and poured onto ice and water (˜4 litres) with vigorous stirring. The resulting grey solid was collected by filtration and washed copiously with water. This was dissolved in ethyl acetate (˜750 ml), dried (sodium sulphate) and evaporated. The residue was purified on an 800 g flash 75 biotage column eluting with 5% and then 10% ethyl acetate in pentane. Fractions containing the product were combined and evaporated to afford the title compound as a colourless crystalline solid. MS (AP+) m/e 419&421 [M+H]⁺.

Description 2

### 1,1-Dimethylethyl 7-{[5-(2-oxo-1-pyrrolidinyl)-2-pyridinyl]oxy}-1,2,4,5-tetrahydro-3H-3-benzazepine-3-carboxylate (D2)

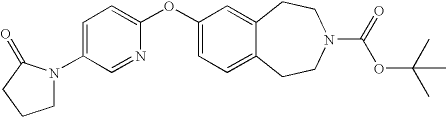

Method A

A mixture of 1,1-dimethylethyl 7-[(5-iodo-2-pyridinyl)oxy]-1,2,4,5-tetrahydro-3H-3-benzazepine-3-carboxylate (D1) (12.5 g; 26.8 mmol), potassium carbonate (12.3 g; 96.5 mmol), 2-pyrrolidinone (4.0 ml; 53.6 mmol), N,N′-dimethyl-1,2-ethanediamine (263 mg; 2.7 mmol) and copper (I) iodide (0.5 g, 2.8 mmol) in dry 1,4-dioxan (200 ml) was heated at reflux for 3 hours. More copper (I) iodide and N,N′-dimethyl-1,2-ethanediamine (10 mol % of each) was added and heating was continued for a further 2 hours. The mixture was allowed to cool and was filtered through celite. The pad was washed with ethyl acetate and the filtrates evaporated to give a pale blue gum. The residue was purified on a 400 g biotage column eluting with 1-1 then 2-1 ethyl acetate-hexane. Fractions containing the product were combined and evaporated to give a white solid (8.33 g; 74%) MS (AP+), m/e 424 [M+H].

Method B

A mixture of copper iodide (136 mg, 0.72 mmol), 1,1-dimethylethyl 7-[(5-bromo-2-pyridinyl)oxy]-1,2,4,5-tetrahydro-3H-3-benzazepine-3-carboxylate (D1a), (3.0 g; 7.2 mmol), pyrrolidinone (1.22 g; 14.3 mmol), potassium carbonate (3.6 g; 25.8 mmol) and N,N′-dimethyl-1,2-ethanediamine (63 mg; 0.72 mmol) in 1,4-dioxan (20 ml) was heated at reflux under argon for 18 hours. The mixture was filtered through Celite and the pad washed with ethyl acetate. The filtrates were evaporated and the residue purified on a 40+m biotage cartridge eluting with a 1-1 to 1-0 gradient of ethyl acetate-pentane. Fractions containing the product were combined and evaporated to afford the title compound as a white crystalline solid. MS (AP+) m/e 424 [M+H]⁺.

Description 3

### 1-[6-(2,3,4,5-Tetrahydro-1H-3-benzazepin-7-yloxy)-3-pyridinyl]-2-pyrrolidinone (D3)

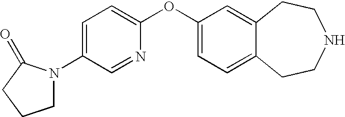

Method A

A solution of 1,1-dimethylethyl 7-{[5-(2-oxo-1-pyrrolidinyl)-2-pyridinyl]oxy}-1,2,4,5-tetrahydro-3H-3-benzazepine-3-carboxylate (D2, method A) (8.1 g; 19.2 mmol) in dichloromethane (50 ml) was added dropwise to a 4M solution of hydrogen chloride in 1,4-dioxan (48 ml; 0.192 mol). When the addition was complete the mixture was stirred for 1 hour at room temperature. The resulting pale yellow solid was collected by filtration and washed with ethyl acetate. This was dissolved in water (50 ml) and the pH adjusted to 14 by the addition of 2M NaOH solution. This was extracted using ethyl acetate (6×70 ml) and the combined extracts washed with brine (100 ml), dried (sodium sulphate) and evaporated to afford the title compound (5.7 g, 92%) MS (AP+), m/e 324 [M+H].

Method B

Trifluoroacetic acid (8 ml) was added to a solution of 1,1-dimethylethyl 7-{[5-(2-oxo-1-pyrrolidinyl)-2-pyridinyl]oxy}-1,2,4,5-tetrahydro-3H-3-benzazepine-3-carboxylate (D2, method B) (2.83 g; 6.7 mmol) in dichloromethane (8 ml) and the mixture stirred at room temperature under argon for 1 hour. The solvent was removed by evaporation and the resulting mixture was purified on 2×10 g SCX (Strong Cation Exchange) cartridges. Fractions containing the product were combined and evaporated to give a colourless gum which solidified on standing. MS (ES+) m/e 324 [M+H]⁺.

Description 4

### 5-Chloro-2-pyrazinamine (D4)

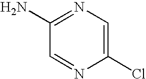

Aminopyrazine (10 g, 105 mmol) was dissolved in dimethylformamide (60 ml) and N-chlorosuccinimide (15.36 g, 115 mmol) was added portionwise under argon at room temperature. After 5 minutes, the temperature rose from 25° C. to 45° C. (care required—exothermic reaction). An ice bath was placed underneath the reaction mixture and the mixture was stirred for 30 minutes and then allowed to warm to room temperature. The mixture was poured onto water and extracted with diethyl ether (x 5). The diethyl ether layer was evaporated under reduced pressure. The product was purified by Biotage column chromatography eluting with 10% ethyl acetate in pentane to afford the title compound (1.40 g). ¹H NMR (CDCl₃) 8.02 (1H, s), 7.76 (1H, s), 4.61 (2H, s).

Description 5

### 2,5-Dichloropyrazine (D5)

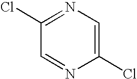

5-Chloro-2-pyrazinamine (D4) (753 mg, 5.81 mmol) was dissolved in concentrated hydrochloric acid (8 ml), cooled in an ice-acetone bath and treated with a solution of sodium nitrite (822 mg, 11.9 mmol) in water (6 ml) dropwise over a period of 1 hour. The mixture was transferred to an ice-water bath and left to stir for 1 hour. The mixture was allowed to warm to room temperature over 2 hours, neutralised by addition of an aqueous 50% sodium hydroxide solution and extracted with dichloromethane (x 4). The dichloromethane layers were combined, dried under magnesium sulfate and evaporated. The resulting residue was purified by Biotage column chromatography eluting with 10% ethyl acetate in pentane to afford the title compound (112 mg). ¹H NMR (CDCl₃) 8.40 (2H, s).

Description 6

### 1,1-Dimethylethyl 7-[(5-chloro-2-pyrazinyl)oxy]-1,2,4,5-tetrahydro-3H-3-benzazepine-3-carboxylate (D6)

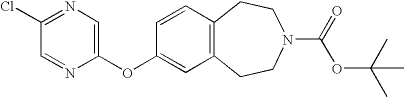

1,1-Dimethylethyl 7-hydroxy-1,2,4,5-tetrahydro-3H-3-benzazepine-3-carboxylate (182 mg, 0.69 mmol: obtainable by the process described in Description 3 from WO 02/40471) was dissolved in dry dimethylformamide (3 ml), cooled in an ice bath and treated with sodium hydride (60% in mineral oil, 29 mg, 0.72 mmol). The mixture was allowed to warm to room temperature over 1 hour. A solution of 2,5-dichloropyrazine (D5) (112 mg, 0.76 mmol) in dimethylformamide was added and the mixture stirred at room temperature for 2 hours and left to stand overnight under argon. The mixture was diluted with water and extracted with ethyl acetate (x 2). The ethyl acetate layers were combined, dried under magnesium sulfate and evaporated. The residue was purified by Biotage column chromatography, eluting with 1:4 ethyl acetate: pentane to afford the title compound (208 mg). MS (ES+) m/e 376 [M+H]⁺.

Description 7

### 1,1-Dimethylethyl 7-{[5-(2-oxo-1-pyrrolidinyl)-2-pyrazinyl]oxy}-1,2,4,5-tetrahydro-3H-3-benzazepine-3-carboxylate (D7)

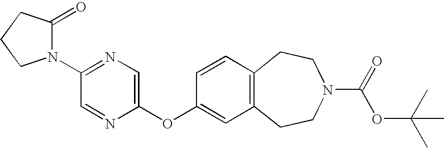

1,1-Dimethylethyl 7-[(5-chloro-2-pyrazinyl)oxy]-1,2,4,5-tetrahydro-3H-3-benzazepine-3-carboxylate (D6) (208 mg, 0.55 mmol), pyrrolidinone (0.08 ml, 1.1 mmol), potassium carbonate (273 mg, 1.98 mmol), copper (I) iodide (32 mg, 0.17 mmol) and N,N-dimethylethylenediamine (0.02 ml, 0.17 mmol) were added together in dry dioxane (5 ml) and heated in a microwave reactor at 140° C. for 20 minutes at high absorption. The mixture was heated under the same conditions for a further 20 minutes followed by heating at 150° C. for 30 minutes at high absorption. A further quantity of pyrrolidinone (2 equivalents), copper (I) iodide (30 mol %) and N,N-dimethylethylenediamine (30 mol %) was added and the mixture was heated at 150° C. for 45 minutes at high absorption. The mixture was then at 150° C. for 1 hour. The mixture was diluted with water and extracted with ethyl acetate (x 3). The ethyl acetate layers were combined, dried under magnesium sulfate and evaporated under reduced pressure. The residue was purified by Biotage column chromatography eluting with 1:1 ethyl acetate:pentane to afford the title compound (126 mg). MS (ES+) m/e 425 [M+H]⁺.

Description 8

### 1-[5-(2,3,4,5-Tetrahydro-1H-3-benzazepin-7-yloxy)-2-pyrazinyl]-2-pyrrolidinone (D8)

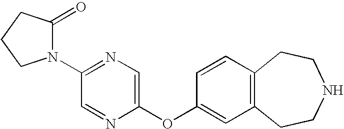

1,1-Dimethylethyl 7-{[5-(2-oxo-1-pyrrolidinyl)-2-pyrazinyl]oxy}-1,2,4,5-tetrahydro-3H-3-benzazepine-3-carboxylate (D7) (126 mg, 0.30 mmol) was dissolved in dichloromethane (2 ml), treated with trifluoroacetic acid (2 ml) and stirred at room temperature under argon for 2 hours. The solvent was removed under reduced pressure and the residue dissolved in methanol and passed down an SCX (Strong cation exchange) column eluting with methanol followed by 2M ammonia/methanol. The basic fractions were combined and evaporated to give the title compound (88 mg). MS (ES+) m/e 325 [M+H]⁺.

Description 9

### 1,1-Dimethylethyl 7-[(2-fluoro-4-nitrophenyl)oxy]-1,2,4,5-tetrahydro-3H-3-benzazepine-3-carboxylate (D9)

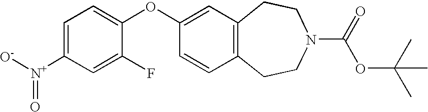

A stirred suspension of 1,1-dimethylethyl 7-hydroxy-1,2,4,5-tetrahydro-3H-3-benzazepine-3-carboxylate (obtainable by the process described in Description 3 from WO 02/40471) (10.0 g, 38.0 mmole) and potassium carbonate (13.0 g, 94.9 mmole) in dimethylformamide was treated with 3,4-difluoronitrobenzene (6.64 g, 41.8 mmole) and the mixture stirred at 130° C. for 18 hours. After cooling to ambient temperature, the reaction mixture was partitioned between ethyl acetate and water. The aqueous layer was extracted (x2) with ethyl acetate and the combined organic extracts washed (x3) with brine, dried (Na₂SO₄) and concentrated in vacuo. The resulting orange oil was purified by column chromatography, eluting with 0-50% ethyl acetate/pentane to afford the title compound; MS (ES+) m/e 303 [M-BOC+H]⁺.

Description 10

### 1,1-Dimethylethyl 7-[(4-amino-2-fluorophenyl)oxy]-1,2,4,5-tetrahydro-3H-3-benzazepine-3-carboxylate (D10)

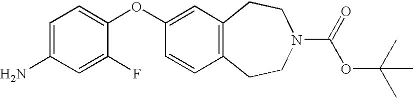

1,1-Dimethylethyl 7-[(2-fluoro-4-nitrophenyl)oxy]-1,2,4,5-tetrahydro-3H-3-benzazepine-3-carboxylate (D9) (17.6 g, 43.8 mmole) in ethanol (125 ml) was hydrogenated in the presence of 10% palladium on charcoal paste for 2 hours. After filtration of the catalyst thorugh celite, the solvent was removed in vacuo to afford the title compound; MS (ES+) m/e 273 [M-BOC+H]⁺.

Description 11

### 1,1-Dimethylethyl 7-[(2-fluoro-4-iodophenyl)oxy]-1,2,4,5-tetrahydro-3H-3-benzazepine-3-carboxylate (D11)

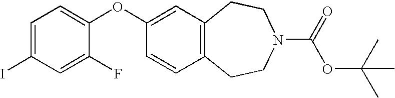

1,1-Dimethylethyl 7-[(4-amino-2-fluorophenyl)oxy]-1,2,4,5-tetrahydro-3H-3-benzazepine-3-carboxylate (D10) (5 g, 13.4 mmole) and iodoform (10.0 g, 26.9 mmole) in dry tetrahydrofuran (100 ml) were treated over 30 minutes with tert-butyl nitrite (3.2 ml, 26.0 mmole) in dry tetrahydrofuran (50 ml). The reaction mixture was stirred at ambient temperature for 1 hour and heated under reflux for 1 hour. After cooling to ambient temperature, the solvent was removed in vacuo and the residue purified by column chromatography eluting with 10% ethyl acetate/pentane to afford the title compound; MS (ES+) m/e 384 [M-BOC+H]⁺.

Description 12

### 7-[(2-Fluoro-4-iodophenyl)oxy]-2,3,4,5-tetrahydro-1H-3-benzazepine (D12)

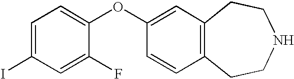

A solution of 1,1-dimethylethyl 7-[(2-fluoro-4-iodophenyl)oxy]-1,2,4,5-tetrahydro-3H-3-benzazepine-3-carboxylate (D11) (4.8 g, 10.0 mmole) in dichloromethane (30 ml) was cooled to 0° C. and treated dropwise with trifluoroacetic acid (20 ml) and the mixture stirred for 3.5 hours at ambient temperature. After evaporation of the solvent, the residue was dissolved in dichloromethane, poured onto ice/0.880 ammonia with stirring and extracted (x3) with dichloromethane. The combined organic extracts were washed with water, dried (Na₂SO₄) and concentrated in vacuo to afford the title compound; MS (ES+) m/e 384 [M+H]⁺.

Description 13

### 3-Cyclobutyl-7-[(2-fluoro-4-iodophenyl)oxy]-2,3,4,5-tetrahydro-1H-3-benzazepine (D13)

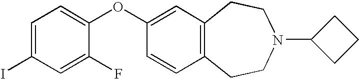

7-[(2-Fluoro-4-iodophenyl)oxy]-2,3,4,5-tetrahydro-1H-3-benzazepine (D12) (3.6 g, 9.3 mmole) in dichloromethane (50 ml) and acetic acid (0.5 ml) was treated with cyclobutanone (1.0 ml, 14.0 mmole) and stirred at ambient temperature for 30 minutes. Sodium triacetoxyborohydride (3.0 g, 14.0 mmole) was added and the mixture stirred for a further 3 hours. 2N Sodium hydroxide solution was added until pH14 was obtained and the mixture was stirred for 30 minutes, diluted with water and extracted (x3) into dichloromethane. The combined organic extracts were washed (x2) with water and brine, dried (Na₂SO₄) and concentrated in vacuo to afford the title compound; MS (ES+) m/e 438 [M+H]⁺.

Description 14

### 1,1-Dimethylethyl 7-[(phenylmethyl)oxy]-1,2,4,5-tetrahydro-3H-3-benzazepine-3-carboxylate (D14)

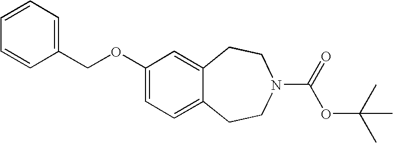

1,1-Dimethylethyl 7-hydroxy-1,2,4,5-tetrahydro-3H-3-benzazepine-3-carboxylate (obtainable by the process described in Description 3 from WO 02/40471) (790 mg, 3 mmol), potassium carbonate (1.24 g, 9 mmol) and catalytic potassium iodide were suspended in 2-butanone (20 ml). Benzyl bromide (536 μl, 4.5 mmol) was added and the mixture heated at reflux for 24 hours. The solids were filtered and then washed with acetone. The filtrate was concentrated in vacuo and the crude oil purified by column chromatography, eluting with a mixture of ethyl acetate and hexane (1:4) to afford the title compound (1.06 g, 100%), ¹H NMR (CDCl₃) 7.44 (5H, m), 7.03 (1H, d, J 8.1 Hz), 6.77 (1H, s), 6.74 (1H, dd, J 8.1 & 2.4 Hz), 3.49 (4H, m), 2.84 (4H, m), 1.48 (9H, s).

Description 15

### 7-[(Phenylmethyl)oxy]-2,3,4,5-tetrahydro-1H-3-benzazepine (D15)

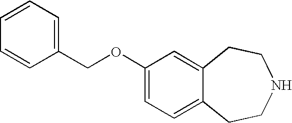

1,1-Dimethylethyl 7-[(phenylmethyl)oxy]-1,2,4,5-tetrahydro-3H-3-benzazepine-3-carboxylate (D14) (1.06 g, 3 mmol) was dissolved in dichloromethane (15 ml) and treated with trifluoroacetic acid (15 ml). The solution was stirred at room temperature for 2 hours, concentrated in vacuo and then twice co-evaporating with dichloromethane. The residue was dissolved in methanol and applied to a SCX (Varian bond-elute, 10 g) and washed with methanol and then a mixture of 0.880 ammonia/methanol. The combined basic fractions were reduced in vacuo and the residue purified by column chromatography (1:9:40 0.880 ammonia:ethanol:dichloromethane) to afford the title compound (702 mg, 93%), MS (ES+) m/e 254 [M+H]⁺.

Description 16

### 3-Cyclobutyl-7-[(phenylmethyl)oxy]-2,3,4,5-tetrahydro-1H-3-benzazepine (D16)

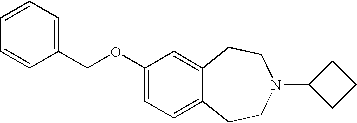

7-[(Phenylmethyl)oxy]-2,3,4,5-tetrahydro-1H-3-benzazepine (D15) (25.3 g, 100 mmol) was dissolved in 2.5% acetic acid in dichloromethane (400 ml) at 0° C. and treated dropwise with cyclobutanone (11.2 ml, 150 mmol). The mixture was stirred for 30 minutes and then sodium triacetoxyborohydride (31.8 g, 150 mmol) was added portion wise. The reaction mixture was stirred at room temperature for 4 hours, basified with saturated sodium carbonate solution and extracted with dichloromethane. The combined extracts were washed with water, brine, dried over anhydrous sodium sulfate and concentrated in vacuo. The crude residue was triturated with hexane and filtered to afford the title product. MS (ES+) m/e 308 [M+H]⁺.

Description 17

### 3-Cyclobutyl-2,3,4,5-tetrahydro-1H-3-benzazepin-7-ol (D17)

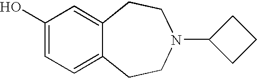

3-Cyclobutyl-7-[(phenylmethyl)oxy]-2,3,4,5-tetrahydro-1H-3-benzazepine (D16) (9.22 g, 30 mmol) was dissolved in ethanol (150 ml) and tetrahydrofuran (50 ml). Palladium (1.5 g, 10% on charcoal paste) was added and the reaction mixture was stirred at room temperature under hydrogen (1 atmosphere) for 5 hours. The reaction mixture was filtered through celite and the filtrate concentrated in vacuo. The crude residue was triturated with diethyl ether and filtered to afford the title product, which was used in subsequent steps without further purification. MS (ES+) m/e 218 [M+H]⁺.

Description 18

### 3-Cyclobutyl-7-[(4-iodophenyl)oxy]-2,3,4,5-tetrahydro-1H-3-benzazepine (D18)

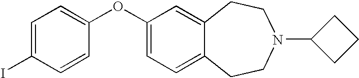

3-Cyclobutyl-2,3,4,5-tetrahydro-1H-3-benzazepin-7-ol (D17) (0.35 g, 1.6 mmole), 4-fluoroiodobenzene (0.23 ml, 1.9 mmole) and cesium carbonate (0.63 g, 1.9 mmole) in dimethylformamide (15 ml) were heated in a microwave reactor at 200° C. at high absorption for 20 minutes. After cooling to ambient temperature, the mixture was partitioned between ethyl acetate and water. The aqueous layer was extracted into ethyl acetate, and the combined organic layers were washed with brine, dried (Na₂SO₄) and concentrated in vacuo. The residue was purified by column chromatography eluting with 2.5% (2M ammonia in methanol)/dichloromethane to afford the title compound; MS (ES+) m/e 420 [M+H]⁺.

Description 19

### 2,3,4,5-Tetrahydro-1H-3-benzazepin-7-ol (D19)

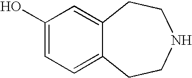

1,1-Dimethylethyl 7-hydroxy-1,2,4,5-tetrahydro-3H-3-benzazepine-3-carboxylate (obtainable by the process described in Description 3 from WO 02/40471) (20.0 g, 76 mmole) in dry dichloromethane (80 ml) was cooled to 0° C. and treated dropwise with trifluoroacetic acid (40 ml). The reaction mixture was stirred for 20 minutes at 0° C. and for 1 hour at ambient temperature after which the solvent was removed in vacuo to afford the title compound as the trifluoroacetate salt; MS (ES+) m/e 164 [M+H]⁺.

Description 20

### 3-Cyclopentyl-2,3,4,6-tetrahydro-1H-3-benzazepin-7-ol (D20)

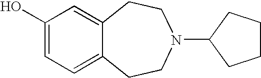

2,3,4,5-Tetrahydro-1H-3-benzazepin-7-ol (D19) (24.6 g, 89 mmole) was suspended in dry dichloromethane (200 ml) and cooled to 0° C. Triethylamine (13.6 ml, 98 mmole) was added and the mixture stirred for 15 minutes. Cyclopentanone (9.4 ml, 107 mmole) was added dropwise and the mixture was allowed to warm to ambient temperature and stirred for 1 hour. After cooling to 0° C., sodium triacetoxyborohydride (22.6 g, 107 mmole) was added portionwise and the mixture stirred at ambient temperature for 72 hours. The solvent was removed in vacuo and the residue partitioned between ethyl acetate and water, filtered through celite and the layers separated. The organic layer was washed with saturated sodium hydrogen carbonate solution, dried (MgSO4)and concentrated in vacuo to afford the title compound; MS (ES+) m/e 232 [M+H]⁺. The residue from the filtration was removed from the celite and dissolved in 2N sodium hydroxide solution. This aqueous layer was neutralised to pH8 by addition of 2N hydrochloric acid, extracted (x3) into ethyl acetate, dried (MgSO₄) and concentrated in vacuo to afford a second crop of the title compound; MS (ES+) m/e 232 [M+H]⁺.

Description 21

### 3-Cyclopentyl-7-[(6-iodo-3-pyridazinyl)oxy]-2,3,4,5-tetrahydro-1H-3-benzazepine (D21)

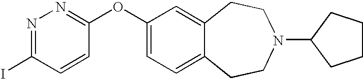

3-Cyclopentyl-2,3,4,5-tetrahydro-1H-3-benzazepin-7-ol (D20) (1.0 g, 4.5 mmole) in dry dimethylformamide (10 ml) was treated with 3,6-diiodopyridazine (J. Med. Chem.; 42; (4); 669; (1999)) (1.8 g, 5.4 mmole) and cesium carbonate (1.8 g, 5.4 mmole) and heated at 100° C. for 2 hours. After cooling to ambient temperature, the solvent was removed in vacuo and the residue partitioned between water and dichloromethane. The organic layer was washed with water and brine, dried (MgSO₄) and evaporated in vacuo. The residue was purified by column chromatography eluting with 0-2% (2M ammonia in methanol)/dichloromethane to afford the title compound; MS (ES+) m/e 436 [M+H]⁺.

Description 22

### 1,1-Dimethylethyl 7-({5-[(methyloxy)carbonyl]-2-pyrazinyl}oxy)-1,2,4,5-tetrahydro-3H-3-benzazepine-3-carboxylate (D22)

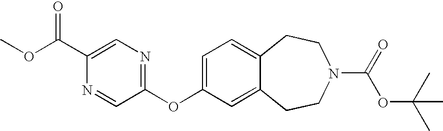

Sodium hydride (800 mg of 60% dispersion; 20 mmol) was added to a solution of 1,1-dimethylethyl 7-hydroxy-1,2,4,5-tetrahydro-3H-3-benzazepine-3-carboxylate (5 g; 19 mmol; obtainable by the process described in Description 3 from WO 02/40471) in dry dimethylformamide (40 ml) and the reaction stirred at room temperature for 45 minutes. Methyl 5-chloro-2-pyrazinecarboxylate (3.9 g; 22.8 mmol) was added and the mixture stirred at room temperature for 18 hours. The mixture was poured into water and extracted with diethyl ether. The extracts were combined, dried (sodium sulphate) and evaporated. The residue was purified by silica column chromatography eluting with 2-1 hexane—ethyl acetate to afford a white powder (4.6 g; 61%) δ(CDCl3) 1.49 (s, 9H), 2.92 (m, 4H), 3.58 (m, 4H), 4.01 (s, 3H), 6.94 (m, 2H), 7.18 (m, H), 8.48 (s, H), 8.84 (s, H).

Description 23

### 5-[(3-{[(1,1-Dimethylethyl)oxy]carbonyl}-2,3,4,5-tetrahydro-1H-3-benzazepin-7-yl)oxy]-2-pyrazinecarboxylic acid (D23)

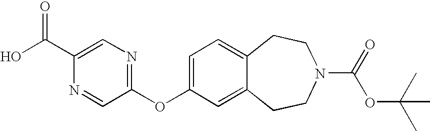

2M Sodium hydroxide solution (18 ml; 36 mmol) was added to a stirring solution of 1,1-dimethylethyl 7-({5-[(methyloxy)carbonyl]-2-pyrazinyl}oxy)-1,2,4,5-tetrahydro-3H-3-benzazepine-3-carboxylate (D22) (5 g; 12.5 mmol) in acetone (80 ml). After 15 minutes the mixture was acidified using 2M hydrochloric acid and this mixture poured into water. The resulting precipitate was collected by filtration and dissolved in ethyl acetate. This was dried (sodium sulphate) and evaporated to give a white powder (4.38 g; 91%) MS (AP+), m/e 386 [M+H].

Description 24

### 1,1-Dimethylethyl 7-{[5-(3-methyl-1,2,4-oxadiazol-5-yl)-2-pyrazinyl]oxy}-1,2,4,5-tetrahydro-3H-3-benzazepine-3-carboxylate (D24)

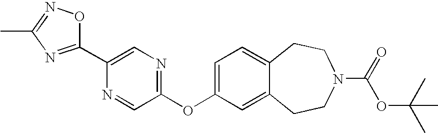

1,1′-(Oxomethanediyl)bis-1H-imidazole (463 mg, 2.9 mmol) was added to a solution of 5-[(3-{[(1,1-dimethylethyl)oxy]carbonyl}-2,3,4,5-tetrahydro-1H-3-benzazepin-7-yl)oxy]-2-pyrazinecarboxylic acid (D23) (1 g; 2.6 mmol) in tetrahydrofuran (15 ml). The mixture was heated at reflux for 1 hour and (1E)-N-hydroxyethanimidamide (385 mg; 5.2 mmol) was added. Heating was continued for a further 18 hours and the mixture diluted with ethyl acetate. This was washed with water, dilute sodium hydroxide solution and brine, dried (sodium sulphate) and evaporated. The residue was purified by silica column chromatography eluting with 2-1 pentane—ethyl acetate to afford a white powder (310 mg; 28%) MS (AP+), m/e 424 [M+H].

Description 25

### 7-{[5-(3-Methyl-1,2,4-oxadiazol-5-yl)-2-pyrazinyl]oxy}-2,3,4,5-tetrahydro-1H-3-benzazepine (D25)

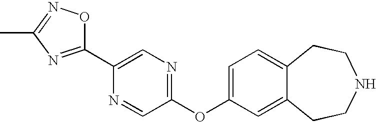

Trifluoroacetic acid (4 ml) was added to a solution of 1,1-dimethylethyl 7-{[5-(3-methyl-1,2,4-oxadiazol-5-yl)-2-pyrazinyl]oxy}-1,2,4,5-tetrahydro-3H-3-benzazepine-3-carboxylate (D24) (300 mg; 0.73 mmol) in dichloromethane (4 ml) and the mixture stirred at room temperature for 30 minutes. The mixture was evaporated and purified on a 10 g SCX ion exchange column eluting with methanol and then 2M ammonia solution in methanol to afford a colourless gum (230 mg; 98%) MS (AP+), m/e 324 [M+H].

Description 26

### 1,1-Dimethylethyl 7-({5-[(methyloxy)carbonyl]-2-pyridinyl}oxy)-1,2,4,5-tetrahydro-3H-3-benzazepine-3-carboxylate (D26)

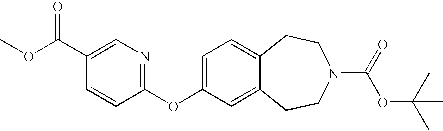

Sodium hydride (540 mg of 60% dispersion; 13.7 mmol) was added to a solution of 1,1-dimethylethyl 7-hydroxy-1,2,4,5-tetrahydro-3H-3-benzazepine-3-carboxylate (3 g; 11.4 mmol; obtainable by the process described in Description 3 from WO 02/40471) in dry dimethylformamide (20 ml) and the reaction stirred at room temperature for 45 minutes. Methyl 6-chloro-3-pyridinecarboxylate (3.9 g; 22.8 mmol) was added and the mixture stirred at 100° C. for 18 hours. The mixture was poured into water and extracted with ethyl acetate. The extracts were combined, dried (sodium sulphate) and evaporated. The residue was purified by silica column chromatography eluting with 3-1 pentane—ethyl acetate to afford a white powder (3.7 g; 82%) MS (AP+), m/e 399 [M+H].

Description 27

### 6-[(3-{[(1,1-Dimethylethyl)oxy]carbonyl}-2,3,4,5-tetrahydro-1H-3-benzazepin-7-yl)oxy]-3-pyridinecarboxylic acid (D27)

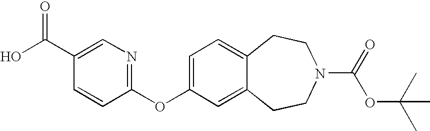

2M Sodium hydroxide solution (14 ml; 28 mmol) was added to a stirring solution of 1,1-dimethylethyl 7-({5-[(methyloxy)carbonyl]-2-pyridinyl}oxy)-1,2,4,5-tetrahydro-3H-3-benzazepine-3-carboxylate (D26) (3.7 g; 9.3 mmol) in acetone (35 ml). After 15 minutes the mixture was acidified using 2M hydrochloric acid and this mixture poured into water. The resulting precipitate was collected by filtration and dissolved in ethyl acetate. This was dried (sodium sulphate) and evaporated to give a white powder (3.07 g; 86%) MS (AP+), m/e 385 [M+H].

Description 28

### 1,1-Dimethylethyl 7-{[5-(3-methyl-1,2,4-oxadiazol-5-yl)-2-pyridinyl]oxy}-1,2,4,5-tetrahydro-3H-3-benzazepine-3-carboxylate (D28)

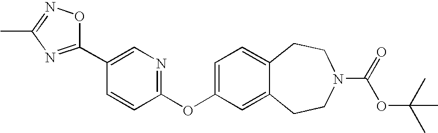

1,1′-(Oxomethanediyl)bis-1H-imidazole (1.42 g, 8.8 mmol) was added to a solution 6-[(3-{[(1,1-dimethylethyl)oxy]carbonyl}-2,3,4,5-tetrahydro-1H-3-benzazepin-7-yl)oxy]-3-pyridinecarboxylic acid (D27) (1.42 g; 8 mmol) in tetrahydrofuran (30 ml). The mixture was heated at reflux for 1 hour and (1E)-N-hydroxyethanimidamide (1.77 g; 24 mmol) was added. Heating was continued for a further 70 hours and the mixture diluted with ethyl acetate. This was washed with water, dilute sodium hydroxide solution and brine, dried (sodium sulphate) and evaporated. The residue was purified by silica column chromatography eluting with 2-1 pentane—ethyl acetate to afford a white powder (1.68 mg; 50%) MS (AP+), m/e 423 [M+H].

Description 29

### 7-{[5-(3-Methyl-1,2,4-oxadiazol-5-yl)-2-pyridinyl]oxy}-2,3,4,5-tetrahydro-1H-3-benzazepine (D29)

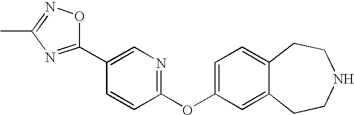

Trifluoroacetic acid (15 ml) was added to a solution of 1,1-dimethylethyl 7-{[5-(3-methyl-1,2,4-oxadiazol-5-yl)-2-pyridinyl]oxy}-1,2,4,5-tetrahydro-3H-3-benzazepine-3-carboxylate (D28) (1.68 g; 4 mmol) in dichloromethane (15 ml) and the mixture stirred at room temperature for 30 minutes. The mixture was evaporated and purified on a 10 g SCX ion exchange column eluting with methanol and then 2M ammonia solution in methanol to afford a colourless gum (230 mg; 98%) δ (CDCl3) 2.47 (3H, s), 2.91-3.00 (8H, m), 6.90 (2H, m), 7.04 (h, m), 7.17 (H, m), 8.33 (H, m), 8.93 (H, m).

### EXAMPLE 1

### 1-{6-[(3-Cyclopentyl-2,3,4,5-tetrahydro-1H-3-benzazepin-7-yl)oxy]-3-pyridinyl}-2-pyrrolidinone (E1)

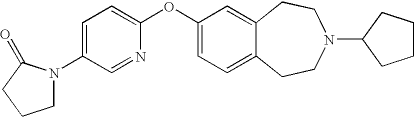

1-[6-(2,3,4,5-Tetrahydro-1H-3-benzazepin-7-yloxy)-3-pyridinyl]-2-pyrrolidinone (D3, method A) (0.10 g, 0.31 mmole) in dry dichloromethane (4 ml) was treated with cyclopentanone (0.033 ml, 0.37 mmole) and stirred for 30 minutes. Sodium triacetoxyborohydride (0.078 g, 0.37 mmole) was added and the mixture stirred overnight at room temp. The crude mixture was diluted with methanol and applied to a SCX (Strong cation exchange) ion exchange cartridge (Varian bond-elute) and washed with methanol and then 2M ammonia in methanol. The basic fractions were concentrated in vacuo. The residue was purified by column chromatography eluting with 1-3% (2M ammonia in methanol)/dichloromethane to afford the title compound as a white solid; MS (ES+) m/e 392 [M+H]⁺. 1 H NMR (CDCl3) d 8.28 (H, d), 8.19 (H, s), 7.10 (H, d), 6.91 (H, d), 6.86-6.86 (2H, m), 3.85 (2H, t), 3.00-2.81 (5H, m), 2.80-2.65 (4H, m), 2.61 (2H, t), 2.20 (2H, quintet), 1.86 (2H, m), 1.73-1.40 (6H, m)

### EXAMPLE 1a

### 1-{6-[(3-Cyclopentyl-2,3,4,5-tetrahydro-1H-3-benzazepin-7-yl)oxy]-3-pyridinyl}-2-pyrrolidinone monohydrochloride salt (E1a)

### Step 1: 1-{6-[(3-Cyclopentyl-2,3,4,5-tetrahydro-1H-3-benzazepin-7-yl)oxy]-3-pyridinyl}-2-2-pyrrolidinone

A mixture of sodium triacetoxyborohydride (2.83 g; 13.4 mmol), 1-[6-(2,3,4,5-tetrahydro-1H-3-benzazepin-7-yloxy)-3-pyridinyl]-2-pyrrolidinone (D3, method B) (2.16 g; 6.7 mmol), cylopentanone (1.18 ml; 13.4 mmol) and glacial acetic acid (0.5 ml) in dichloromethane (25 ml) was stirred at room temperature for 18 hours. The mixture was purified on 2×10 g SCX (strong cation exchange) cartridges. The basic fractions were combined and evaporated to give a white solid which was purified on a 40+m biotage cartridge eluting with a 0-5% gradient of 2M ammonia in methanol in dichloromethane. Fractions containing the product were combined and evaporated to give a colourless gum. This material was dissolved in methanol and purified on 2×10 g SCX (strong cation exchange) cartridges. The basic fractions were combined and evaporated to give a colourless gum. MS (AP+) m/e 392 [M+H]⁺.

### Step 2: 1-{6-[(3-Cyclopentyl-2,3,4,5-tetrahydro-1H-3-benzazepin-7-yl)oxy]-3-pyridinyl}-2-pyrrolidinone monohydrochloride salt

1M Hydrogen chloride in diethylether (6.6 ml; 6.6 mmol) was added to solution of 1-{6-[(3-cyclopentyl-2,3,4,5-tetrahydro-1H-3-benzazepin-7-yl)oxy]-3-pyridinyl}-2-pyrrolidinone (2.46 g; 6.3 mmol) in methanol (20 ml) and the mixture stirred at room temperature under argon for 90 minutes. The solvent was removed by evaporation to give a cream solid. This was suspended in ethyl acetate (100 ml) and heated to ˜100° C. Methanol (˜60 ml) was added until a clear solution was obtained. The azeotrope was removed by distillation until a turbid solution was obtained. The mixture was allowed to cool slowly under argon and stirred for 18 hours. The resulting solid was collected by filtration, washed with ethyl acetate and dried in a vacuum oven at 40° C. for 4 days to afford the title compound as a white powder. MS (ES+) m/e 392 [M+H]⁺.

¹H NMR (δ⁶-DMSO) δ 11.25 (H, br), 8.34 (H, s), 8.21 (H, d), 7.23 (H, d), 7.05 (H, d), 6.99 (H, s), 6.92 (H, d) 3.83 (2H, t), 3.64 (3H, m), 3.46 (2H, m), 2.99 (4H, m), 2.50 (2H, m), 2.01-2.10 (4H, m), 1.88 (2H, m), 1.73 (2H, m), 1.54 (2H, m).

### EXAMPLE 2

### 1-{5-[(3-Cyclopentyl-2,3,4,5-tetrahydro-1H-3-benzazepin-7-yl)oxy]-2-pyrazinyl}-2-pyrrolidinone (E2)

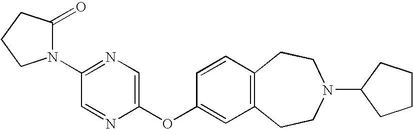

1-[5-(2,3,4,5-Tetrahydro-1H-3-benzazepin-7-yloxy)-2-pyrazinyl]-2-pyrrolidinone (D8) (60 mg, 0.19 mmol) was dissolved in dichloromethane (3 ml) and treated with cyclopentanone (0.03 ml, 0.38 mmol) and acetic acid (2 drops). The mixture was stirred at room temperature for 5 minutes under argon. Sodium triacetoxyborohydride (81 mg, 0.38 mmol) was added and the mixture was left to stir for 30 minutes. The reaction mixture was diluted with methanol and passed down an SCX (Strong cation exchange) column eluting with methanol followed by 2M ammonia/methanol. The basic fractions were combined and evaporated under reduced pressure. The product was purified by column chromatography eluting with 5% (2M ammonia in methanol)—95% dichloromethaneto give the title compound (61 mg). MS(ES+) m/e 393 [M+H]⁺. ¹H NMR (CDCl₃) 9.25 (1H, s), 8.10 (1H, s), 7.12-7.09 (1H, d), 6.87-6.85 (2H, m), 4.06-4.02 (2H, t), 2.92-2.86 (5H, m), 2.72-2.64 (6H, m), 2.21-2.15 (2H, m), 1.87-1.85 (2H, m), 1.58-1.47 (6H, m).

### EXAMPLE 3

### 1-{4-[(3-Cyclobutyl-2,3,4,5-tetrahydro-1H-3-benzazepin-7-yl)oxy]-3-fluorophenyl}-3-methyl-2-imidazolidinone (E3)

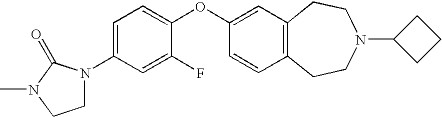

A mixture of 3-cyclobutyl-7-[(2-fluoro-4-iodophenyl)oxy]-2,3,4,5-tetrahydro-1H-3-benzazepine (D12) (0.30 g, 0.69 mmole), 1-methyl-2-imidazolidinone (0.14 g, 1.37 mmole), copper (I) iodide (0.040 g, 0.21 mmole), potassium carbonate (0.34 g, 2.5 mmole) and N,N′-dimethyl-1,2-ethanediamine (0.018 g, 0.20 mmole) in dry 1,4-dioxane (5 ml) was heated in a microwave reactor at 140° C. at high absorption for 1 hour. After cooling to ambient temperature, the reaction mixture was partitioned between water and ethyl acetate. The organic layer was further extracted into ethyl acetate and the combined organic extracts were washed with brine, dried (Na₂SO₄) and concentrated in vacuo. The residue was purified by column chromatography eluting with 2% (2M ammonia in methanol)/dichloromethane to afford the title compound; MS (ES+) m/e 410 [M+H]⁺.

### EXAMPLE 4

### 1-{4-[(3-Cyclobutyl-2,3,4,5-tetrahydro-1H-3-benzazepin-7-yl)oxy]phenyl}-2-pyrrolidinone (E4)

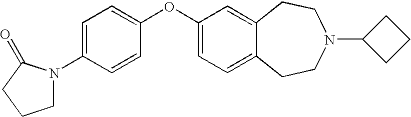

A mixture of 3-cyclobutyl-7-[(4-iodophenyl)oxy]-2,3,4,5-tetrahydro-1H-3-benzazepine (D18) (0.27 g, 0.65 mmole), 2-pyrrolidinone (0.11 g, 1.3 mmole), copper (I) iodide (0.040 g, 0.21 mmole), potassium carbonate (0.32 g, 2.3 mmole) and N,N′-dimethyl-1,2-ethanediamine (0.02 g, 0.20 mmole) in dry 1,4-dioxane (5 ml) was heated in a microwave reactor at 140° C. at high absorption for 1 hour. After cooling to ambient temperature, the reaction mixture was partitioned between water and ethyl acetate. The organic layer was further extracted into ethyl acetate and the combined organic extracts were washed with brine, dried (Na2SO4) and concentrated in vacuo. The residue was purified by column chromatography eluting with 2.5% (2M ammonia in methanol)/dichloromethane to afford the title compound; MS (ES+) m/e 377 [M+H]⁺.

### EXAMPLE 5

### 3-{6-[(3-Cyclopentyl-2,3,4,5-tetrahydro-1H-3-benzazepin-7-yl)oxy]-3-pyridazinyl}-1,3-oxazolidin-2-one (E5)

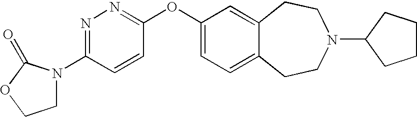

3-Cyclopentyl-7-[(6-iodo-3-pyridazinyl)oxy]-2,3,4,5-tetrahydro-1H-3-benzazepine (D21) (0.20 g, 0.46 mmole), 1,3-oxazolidin-2-one (0.079 g, 0.92 mmole), copper (I) iodide (0.027 g, 0.14 mmole), potassium carbonate (0.23 g, 1.7 mmole) and N,N′-dimethyl-1,2-ethanediamine (0.020 ml, 0.14 mmole) in dry 1,2-dioxane (4 ml) were heated in a microwave reactor at 140° C. at high absorption for 15 minutes. After cooling to ambient temperature, the crude mixture was applied to a SCX ion exchange cartridge (Varian bond-elute) and washed with methanol and then 2M ammonia in methanol. The basic fractions were reduced in vacuo to afford the title compound; MS (ES+) m/e 395 [M+H]⁺.

### EXAMPLE 6

### 1-{6-[(3-Cyclopentyl-2,3,4,5-tetrahydro-1H-3-benzazepin-7-yl)oxy]-3-pyridazinyl}-2-pyrrolidinone (E6)

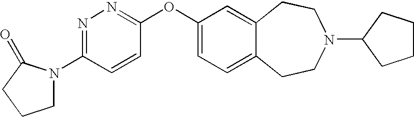

3-Cyclopentyl-7-[(6-iodo-3-pyridazinyl)oxy]-2,3,4,5-tetrahydro-1H-3-benzazepine (D21) (0.20 g, 0.46 mmole), 2-pyrrolidinone (0.078 g, 0.92 mmole), copper (I) iodide (0.027 g, 0.14 mmole), potassium carbonate (0.23 g, 1.7 mmole) and N,N′-dimethyl-1,2-ethanediamine (0.020 ml, 0.14 mmole) in dry 1,2-dioxane (4 ml) were heated in a microwave reactor at 140° C. at high absorption for 30 minutes. After cooling to ambient temperature, the crude mixture was applied to a SCX ion exchange cartridge (Varian bond-elute) and washed with methanol and then 2M ammonia in methanol. The basic fractions were reduced in vacuo and purified by column chromatography eluting with 0-2% (2M ammonia in methanol)/dichloromethane to afford the title compound; MS (ES+) m/e 393 [M+H]⁺.

### EXAMPLE 7

### 3-Cyclopentyl-7-{[5-(3-methyl-1,2,4-oxadiazol-5-yl)-2-pyrazinyl]oxy}-2,3,4,5-tetrahydro-1H-3-benzazepine (E7)

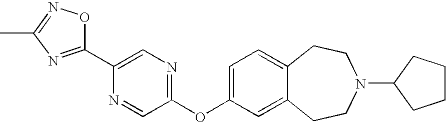

Sodium triacetoxyborohydride (93 mg; 0.44 mmol) was added to a stirring mixture of 7-{[5-(3-methyl-1,2,4-oxadiazol-5-yl)-2-pyrazinyl]oxy}-2,3,4,5-tetrahydro-1H-3-benzazepine (D25) (130 mg; 0.4 mmol), cyclopentanone (0.07 ml; 0.8 mmol) and glacial acetic acid (1 drop) in dichloromethane (4 ml). After stirring at room temperature for 90 minutes the mixture was diluted with methanol and purified on a 10 g SCX ion exchange column eluting with methanol and the 2M ammonia solution in methanol. The residue was then further purified by silica column chromatography eluting with 3-97 2M ammonia in methanol in dichloromethane to afford a white powder (98 mg; 63%) MS (AP+), m/e 392 [M+H].

### EXAMPLE 8

### 3-Cyclobutyl-7-{[5-(3-methyl-1,2,4-oxadiazol-5-yl)-2-pyridinyl]oxy}-2,3,4,5-tetrahydro-1H-3-benzazepine (E8)

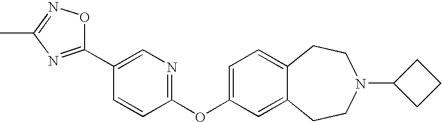

Sodium triacetoxyborohydride (196 mg; 0.93 mmol) was added to stirring mixture of 7-{[5-(3-methyl-1,2,4-oxadiazol-5-yl)-2-pyridinyl]oxy}-2,3,4,5-tetrahydro-1H-3-benzazepine (D29) (150 mg; 0.47 mmol), cyclobutanone (0.11 ml; 1.4 mmol) and glacial acetic acid (1 drop) in dichloromethane (4 ml). After stirring at room temperature for 90 minutes the mixture was diluted with methanol and purified on a 10 g SCX ion exchange column eluting with methanol and the 2M ammonia solution in methanol. The residue was then further purified by silica column chromatography eluting with 3-97 2M ammonia in methanol in dichloromethane to afford a white powder (122 mg; 69%) MS (AP+), m/e 377 [M+H].

### EXAMPLE 9

### 3-Cyclopentyl-7-{[5-(3-methyl-1,2,4-oxadiazol-5-yl)-2-pyridinyl]oxy}-2,3,4,5-tetrahydro-1H-3-benzazepine (E9)

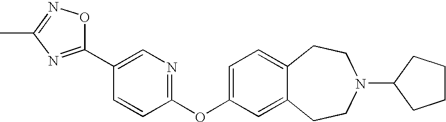

Sodium triacetoxyborohydride (196 mg; 0.93 mmol) was added to stirring mixture of 7-{[5-(3-methyl-1,2,4-oxadiazol-5-yl)-2-pyridinyl]oxy}-2,3,4,5-tetrahydro-1H-3-benzazepine (D29) (150 mg; 0.47 mmol), cyclopentanone (0.12 ml; 1.4 mmol) and glacial acetic acid (1 drop) in dichloromethane (4 ml). After stirring at room temperature for 90 minutes the mixture was diluted with methanol and purified on a 10 g SCX ion exchange column eluting with methanol and the 2M ammonia solution in methanol. The residue was then further purified by silica column chromatography eluting with 3-97 2M ammonia in methanol in dichloromethane to afford a white powder (109 mg; 59%) MS (AP+), m/e 391 [M+H].

Biological Data

A membrane preparation containing histamine H3 receptors may be prepared in accordance with the following procedures:

(i) Generation of Histamine H3 Cell Line

DNA encoding the human histamine H3 gene (Huvar, A. et al. (1999) Mol. Pharmacol. 55(6), 1101-1107) was cloned into a holding vector, pCDNA3.1 TOPO (InVitrogen) and its cDNA was isolated from this vector by restriction digestion of plasmid DNA with the enzymes BamH1 and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos. 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μg ml⁻¹. Colonies containing the re-ligated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen).

CHO K1 cells previously transfected with the pSwitch regulatory plasmid (InVitrogen) were seeded at 2×10e6 cells per T75 flask in Complete Medium, containing Hams F12 (GIBCOBRL, Life Technologies) medium supplemented with 10% v/v dialysed foetal bovine serum, L-glutamine, and hygromycin (100 μg ml⁻¹), 24 hours prior to use. Plasmid DNA was transfected into the cells using Lipofectamine plus according to the manufacturers guidelines (InVitrogen). 48 hours post transfection cells were placed into complete medium supplemented with 500 μg ml⁻¹ Zeocin™.

10-14 days post selection 10 nM Mifepristone (InVitrogen), was added to the culture medium to induce the expression of the receptor. 18 hours post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone). Approximately 1×10e7 cells were examined for receptor expression by staining with a rabbit polyclonal antibody, 4a, raised against the N-terminal domain of the histamine H3 receptor, incubated on ice for 60 minutes, followed by two washes in sorting medium. Receptor bound antibody was detected by incubation of the cells for 60 minutes on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μg ml⁻¹ Zeocin™ and allowed to expand before reanalysis for receptor expression via antibody and ligand binding studies. One clone, 3H3, was selected for membrane preparation.

(ii) Membrane Preparation from Cultured Cells

All steps of the protocol are carried out at 4° C. and with pre-cooled reagents. The cell pellet is resuspended in 10 volumes of homogenisation buffer (50 mM N-2-hydroxyethylpiperazine-N′-2-ethanesulfonic acid (HEPES), 1 mM ethylenediamine tetra-acetic acid (EDTA), pH 7.4 with KOH, supplemented with 10e-6M leupeptin (acetyl-leucyl-leucyl-arginal; Sigma L2884), 25 μg/ml bacitracin (Sigma B0125), 1 mM phenylmethylsulfonyl fluoride (PMSF) and 2×10 e-6M pepstain A (Sigma)). The cells are then homogenised by 2×15 second bursts in a 1 litre glass Waring blender, followed by centrifugation at 500 g for 20 minutes. The supernatant is then spun at 48,000 g for 30 minutes. The pellet is resuspended in homogenisation buffer (4× the volume of the original cell pellet) by vortexing for 5 seconds, followed by homogenisation in a Dounce homogeniser (10-15 strokes). At this point the preparation is aliquoted into polypropylene tubes and stored at −80° C.

Compounds of the invention may be tested for in vitro biological activity in accordance with the following assays:

(I) Histamine H3 Functional Antagonist Assay (Method A)

For each compound being assayed, in a solid white 384 well plate, is added: (a) 5 μl of test compound diluted to the required concentration in 10% DMSO (or 5 μl 10% DMSO as a control); and

(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH7.4 NaOH) to give a final volume of 30 μl which contains 5 μg protein and 0.25 mg bead per well, incubating at 4° C. for 30 minutes on a roller and, just prior to addition to the plate, adding 10 μM final concentration of guanosine 5′ diphosphate (GDP) (Sigma; diluted in assay buffer).

The plates were then incubated at room temperature for 30 minutes on a shaker followed by addition of:

(c) 15 μl 0.38 nM [³⁵S]-GTPγS (Amersham; Radioactivity concentration=37 MBq/ml; Specific activity=1160 Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

(II) Histamine H3 Functional Antagonist Assay (Method B)

For each compound being assayed, in a solid white 384 well plate, is added:

(a) 0.5 μl of test compound diluted to the required concentration in DMSO (or 0.5 μl DMSO as a control);

(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH7.4 NaOH) to give a final volume of 30 μl which contains 5 μg protein and 0.25 mg bead per well, incubating at room temperature for 60 minutes on a roller and, just prior to addition to the plate, adding 10 μM final concentration of guanosine 5═ diphosphate (GDP) (Sigma; diluted in assay buffer);

(c) 15 μl 0.38 nM [³⁵S]-GTPγS (Amersham; Radioactivity concentration=37 MBq/ml; Specific activity=1160 Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

Results

The compounds of examples E1 to E12 were tested in the histamine H3 functional antagonist assay (method A). All compounds exhibited antagonism in this assay as shown in the following table. The results are expressed as functional pKᵢ (fpKᵢ) values. A functional pKi is the negative logarithm of the antagonist equilibrium dissociation constant as determined in the H3 functional antagonist assay using membrane prepared from cultured H3 cells. The results given are averages of a number of experiments.

| Compound | Antagonism (fpKᵢ) |
|---|---|
| 1 | 10.1 |
| 2 | 10.1 |
| 3 | 10.3 |
| 4 | 9.8 |
| 5 | 10.3 |
| 6 | 9.9 |
| 7 | 9.9 |
| 8 | 10.0 |
| 9 | 9.6 |

The compounds of examples E1-E3 were also tested in the histamine H3 functional antagonist assay (method B). All compounds exhibited antagonism in this assay as shown in the following table. Again, the results are expressed as functional pKᵢ (fpKᵢ) values and are averages of a number of experiments.

| Compound | Antagonism (fpKᵢ) |
|---|---|
| 1 | 9.8 |
| 2 | 9.8 |
| 3 | 9.8 |

What is claimed is:

1.-18. (canceled)

19. A compound which is selected from the group consisting of:
1-{6-[(3-cyclopentyl-2,3,4,5-tetrahydro-1H-3-benzazepin-7-yl)oxy]-3-pyridinyl}-2-pyrrolidinone;
1-{5-[(3-cyclopentyl-2,3,4,5-tetrahydro-1H-3-benzazepin-7-yl)oxy]-2-pyrazinyl}-2-pyrrolidinone;
1-{4-[(3-cyclobutyl-2,3,4,5-tetrahydro-1H-3-benzazepin-7-yl)oxy]-3-fluorophenyl}-3-methyl-2-imidazolidinone;
1-{4-[(3-cyclobutyl-2,3,4,5-tetrahydro-1H-3-benzazepin-7-yl)oxy]phenyl}-2-pyrrolidinone;
3-{6-[(3-cyclopentyl-2,3,4,5-tetrahydro-1H-3-benzazepin-7-yl)oxy]-3-pyridazinyl}-1,3-oxazolidin-2-one;
1-{6-[(3-cyclopentyl-2,3,4,5-tetrahydro-1H-3-benzazepin-7-yl)oxy]-3-pyridazinyl}-2-pyrrolidinone;
3-cyclopentyl-7-{[5-(3-methyl-1,2,4-oxadiazol-5-yl)-2-pyrazinyl]oxy}-2,3,4,5-tetrahydro-1H-3-benzazepine;
3-cyclobutyl-7-{[5-(3-methyl-1,2,4-oxadiazol-5-yl)-2-pyridinyl]oxy}-2,3,4,5-tetrahydro-1H-3-benzazepine;
3-cyclopentyl-7-{[5-(3-methyl-1,2,4-oxadiazol-5-yl)-2-pyridinyl]oxy}-2 3,4,5-tetrahydro-1H-3-benzazepine;
or a pharmaceutically acceptable salt thereof.

20. A pharmaceutical composition which comprises a compound as defined in claim 19 or a pharmaceutically acceptable salt thereof and a pharmaceutically acceptable carrier or excipient.

21. A method of treatment of neurological diseases which comprises administering to a host in need thereof an effective amount of a compound as defined in claim 19 or a pharmaceutically acceptable salt thereof.

22. A process for the preparation of 1-{6-[(3-cyclopentyl-2,3,4,5-tetrahydro-1H-3-benzazepin-7-yl)oxy]-3-pyridinyl}-2-pyrrolidinone, which process comprises:
(a) reacting a compound of formula (VI) 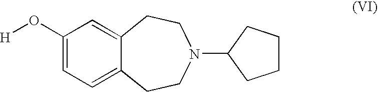
with a compound of formula 3-(pyrrolidin-2-one)-pyridin-6-yl-L¹, wherein L¹ represents a leaving group or an optionally activated hydroxyl group; or
(b) reacting a compound of formula (VII) 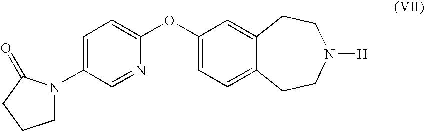
with a compound of formula cyclopentyl-L², wherein L² represents a leaving group; or
(c) reacting a compound of formula (VII) as defined above, with cyclopentanone; or
(d) reacting a compound of formula (VIII) 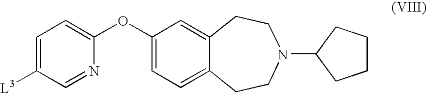
wherein L³ represents a leaving group with pyrrolidinone; or
(f) deprotecting a protected compound to produce 1-{6-[(3-cyclopentyl-2,3,4,5-tetrahydro-1H-3-benzazepin-7-yl)oxy]-3-pyridinyl}-2-pyrrolidinone.

23. A process for the preparation of 1-{5-[(3-cyclopentyl-2,3,4,5-tetrahydro-1H-3-benzazepin-7-yl)oxy]-2-pyrazinyl}-2-pyrrolidinone, which process comprises:
(a) reacting a compound of formula (VI) 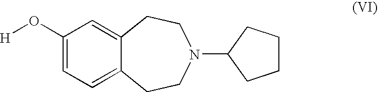
with a compound of formula 5-(pyrrolidin-2-one)-pyrazin-2-yl-L¹, wherein L¹ represents a leaving group or an optionally activated hydroxyl group; or
(b) reacting a compound of formula (IX) 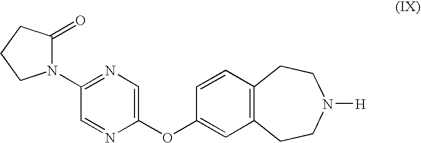
with a compound of formula cyclopentyl-L², wherein L² represents a leaving group; or
(c) reacting a compound of formula (IX) as defined above, with cyclopentanone; or
(d) reacting a compound of formula (X) 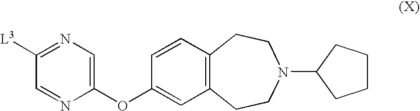
wherein L³ represents a leaving group with pyrrolidinone; or
(f) deprotecting a protected compound to produce 1-{5-[(3-cyclopentyl-2,3,4,5-tetrahydro-1H-3-benzazepin-7-yl)oxy]-2-pyrazinyl}-2-pyrrolidinone.

24. A process for the preparation of 1-{4-[(3-cyclobutyl-2,3,4,5-tetrahydro-1H-3-benzazepin-7-yl)oxy]-3-fluorophenyl}-3-methyl-2-imidazolidinone, which process comprises:
(a) reacting a compound of formula (XI) 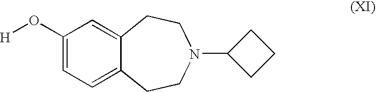
with a compound of formula 4-(3-methyl-imidazolidin-2-one)-2-flurophenyl-L¹, wherein L¹ represents a leaving group or an optionally activated hydroxyl group; or
(b) reacting a compound of formula (XII) 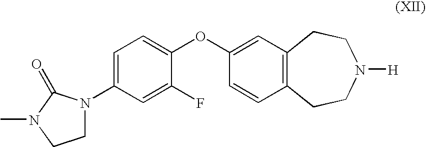
with a compound of formula cyclobutyl-L², wherein L² represents a leaving group; or
(c) reacting a compound of formula (XII) as defined above, with cyclobutanone; or
(d) reacting a compound of formula (XIII) 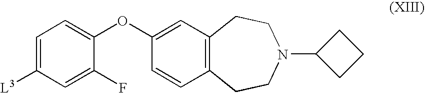
wherein L³represents a leaving group with 1-methyl-2-imidazolidinone; or
(f) deprotecting a protected compound to produce 1-{4-[(3-cyclobutyl-2,3,4,5-tetrahydro-1H-3-benzazepin-7-yl)oxy]-3-fluorophenyl}-3-methyl-2-imidazolidinone.

25. A process for the preparation of 1-{4-[(3-cyclobutyl-2,3,4,5-tetrahydro-1H-3-benzazepin-7-yl)oxy]phenyl}-2-pyrrolidinone, which process comprises:
(a) reacting a compound of formula (XI) 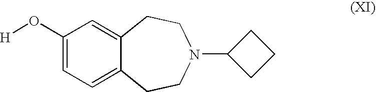
with a compound of formula pyrrolidin-2-one-phenyl-L¹, wherein L¹ represents a leaving group or an optionally activated hydroxyl group; or
(b) reacting a compound of formula (XIV) 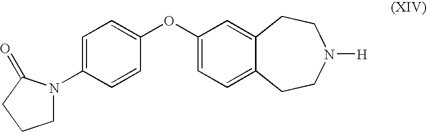
with a compound of formula cyclobutyl-L², wherein L² represents a leaving group; or
(c) reacting a compound of formula (XIV) as defined above, with cyclobutanone; or
(d) reacting a compound of formula (XV) 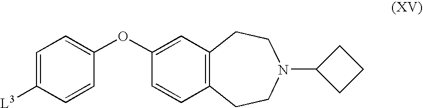
wherein L³ represents a leaving group with 2-pyrrolidinone; or
(f) deprotecting a protected compound to produce 1-{4-[(3-cyclobutyl-2,3,4,5-tetrahydro-1H-3-benzazepin-7-yl)oxy]phenyl}-2-pyrrolidinone.

26. A process for the preparation of 3-{6-[(3-cyclopentyl-2,3,4,5-tetrahydro-1H-3-benzazepin-7-yl)oxy]-3-pyridazinyl}-1,3-oxazolidin-2-one, which process comprises:
(a) reacting a compound of formula (VI) 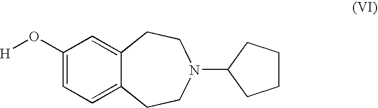
with a compound of formula 3-(1,3-oxazolidin-2-one)-pyridazin-6-yl-L¹, wherein L¹ represents a leaving group or an optionally activated hydroxyl group; or
(b) reacting a compound of formula (XVI) 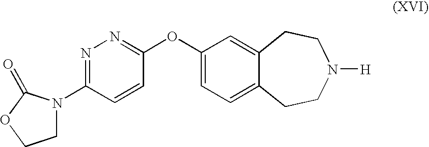
with a compound of formula cyclopentyl-L², wherein L² represents a leaving group; or
(c) reacting a compound of formula (XVI) as defined above, with cyclopentanone; or
(d) reacting a compound of formula (XVII) 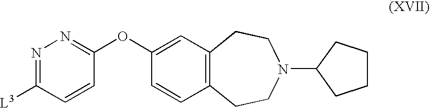
wherein L³ represents a leaving group with 1,3-oxazolidin-2-one; or
(f) deprotecting a protected compound to produce 3-{6-[(3-cyclopentyl-2,3,4,5-tetrahydro-1H-3-benzazepin-7-yl)oxy]-3-pyridazinyl}-1,3-oxazolidin-2-one.

27. A process for the preparation of 1-{6-[(3-cyclopentyl-2,3,4,5-tetrahydro-1H-3-benzazepin-7-yl)oxy]-3-pyridazinyl}-2-pyrrolidinone, which process comprises:
(a) reacting a compound of formula (VI) 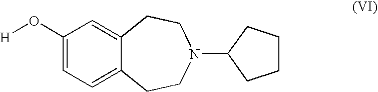
with a compound of formula 3-(pyrrolidinone-2-one)-pyridazin-6-yl-L¹, wherein L¹ represents a leaving group or an optionally activated hydroxyl group; or
(b) reacting a compound of formula (XVIII) 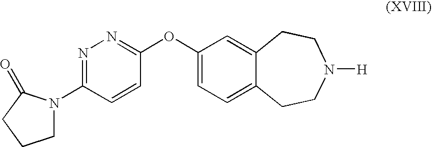
with a compound of formula cyclopentyl-L², wherein L² represents a leaving group; or
(c) reacting a compound of formula (XVIII) as defined above, with cyclopentanone; or
(d) reacting a compound of formula (XVII) 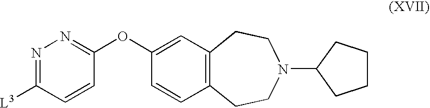
wherein L³ represents a leaving group with pyrrolidinone; or
(f) deprotecting a protected compound to produce 1-{6-[(3-cyclopentyl-2,3,4,5-tetrahydro-1H-3-benzazepin-7-yl)oxy]-3-pyridazinyl}-2-pyrrolidinone.

28. A process for the preparation of 3-cyclopentyl-7-{[5-(3-methyl-1,2,4-oxadiazol-5-yl)-2-pyrazinyl]oxy}-2,3,4,5-tetrahydro-1H-3-benzazepine, which process comprises:
(a) reacting a compound of formula (VI) 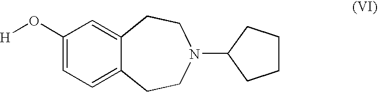
with a compound of formula 5-(3-methyl-1,2,4-oxadiazol-2-yl)-pyrazin-2-yl-L¹, wherein L¹ represents a leaving group or an optionally activated hydroxyl group; or
(b) reacting a compound of formula (XIX) 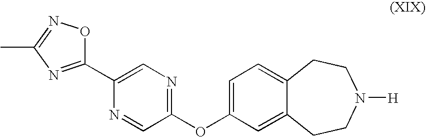
with a compound of formula cyclopentyl-L², wherein L² represents a leaving group; or
(c) reacting a compound of formula (XIX) as defined above, with cyclopentanone; or
(d) reacting a compound of formula (XX) 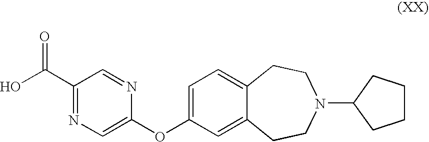
with 1,1′-(Oxomethanediyl)bis-1H-imidazole; or
(f) deprotecting a protected compound to produce 3-cyclopentyl-7-{[5-(3-methyl-1,2,4-oxadiazol-5-yl)-2-pyrazinyl]oxy}-2,3,4,5-tetrahydro-1H-3-benzazepine.

29. A process for the preparation of 3-cyclobutyl-7-{[5-(3-methyl-1,2,4-oxadiazol-5-yl)-2-pyridinyl]oxy}-2,3,4,5-tetrahydro-1H-3-benzazepine, which process comprises:
(a) reacting a compound of formula (XI) 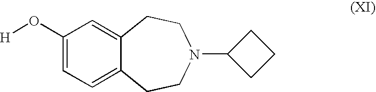
with a compound of formula 5-(3-methyl-1,2,4-oxadiazol-2-yl)-pyridiin-2-yl-L¹, wherein L¹ represents a leaving group or an optionally activated hydroxyl group; or
(b) reacting a compound of formula (XXI) 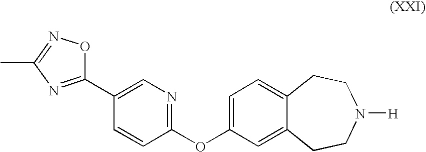
with a compound of formula cyclobutyl-L², wherein L² represents a leaving group; or
(c) reacting a compound of formula (XXI) as defined above, with cyclobutanone; or
(d) reacting a compound of formula (XXII) 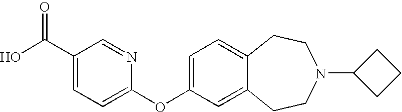
with 1,1′-(oxomethanediyl)bis-1H-imidazole; or
(f) deprotecting a protected compound to produce 3-cyclobutyl-7-{[5-(3-methyl-1,2,4-oxadiazol-5-yl )-2-pyridinyl]oxy}-2,3,4,5-tetrahydro-1H-3-benzazepine.

30. A process for the preparation of 3-cyclopentyl-7-{[5-(3-methyl-1,2,4-oxadiazol-5-yl)-2-pyridinyl]oxy}-2,3,4,5-tetrahydro-1H-3-benzazepine, which process comprises:
(a) reacting a compound of formula (VI) 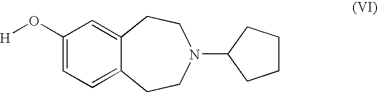
with a compound of formula 5-(3-methyl-1,2,4-oxadiazol-2-yl)-pyridin-2-yl-L¹, wherein L¹ represents a leaving group or an optionally activated hydroxyl group; or
(b) reacting a compound of formula (XXI) 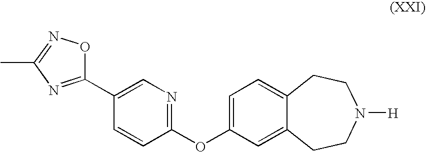
with a compound of formula cyclopentyl-L², wherein L² represents a leaving group; or
(c) reacting a compound of formula (XXI) as defined above, with cyclopentanone; or
(d) reacting a compound of formula (XXIII) 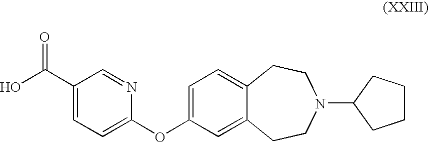
with 1,1′-(oxomethanediyl)bis-1H-imidazole; or
(f) deprotecting a protected compound to produce 3-cyclopentyl-7-{[5-(3-methyl-1,2,4-oxadiazol-5-yl)-2-pyridinyl]oxy}-2,3,4,5-tetrahydro-1H-3-benzazepine.
